(19) United States
(12) Patent Application Publication
(10) Pub. No.: US 2004/0055610 A1
(43) Pub. Date: Mar. 25, 2004

(54) Detection of implanted wireless energy receiving device

(76) Inventor: Peter Forsell, Zug (CH)

Correspondence Address: NIXON & VANDERHYE P.C., 8th Floor, 1100 North Glebe Road, Arlington, VA, 22201, US

(21) Appl. No.: 10/253,897

(22) Filed: Sep. 25, 2002

(51) Int. Cl. A61B 19/00

(52) U.S. Cl. 128/899

(57) ABSTRACT

An apparatus is disclosed for detecting a wireless energy receiving device subcutaneously implanted in a patient's body to enable accurate positioning of a wireless energy transmission device outside the patient's body relative to the energy receiving device. Also disclosed is a method for detecting the wireless energy receiving device whereby an energy transmission device can be positioned to efficiently transmit wireless energy to the implanted energy receiving device. The apparatus includes a magnetic device that is subcutaneously implanted in the patient adjacent to the energy receiving device to emit a local magnetic field through the patient's skin adjacent to the energy receiving device. A magnetic detector movable externally along the patient's body is capable of detecting the local magnetic field emitted by the magnetic device. This allows the energy transmission device to be located for the efficient transmission of wireless energy to the implanted energy receiving device. Alternatively, the apparatus can include a magnetic detector subcutaneously implanted in the patient at the energy receiving device and an exterior magnetic device movable along the patient's skin to emit a magnetic field that is detected by the implanted magnetic detector. Preferably, the magnetic detector includes a semiconductor circuit that is comprised of at least one Hall element. The magnetic device may be a solenoid or a permanent magnet. The energy receiving device can be used to control a restriction device implant designed for treating reflux disease, urinary incontinence, impotence, anal incontinence or obesity.

### FIELD OF THE INVENTION

The present invention relates to apparatuses and methods for detecting a wireless energy receiving device subcutaneously implanted in a patient to enable accurate positioning of an exterior wireless energy transmission device. The present invention also relates to surgical methods for providing a patient with such an apparatus.

### BACKGROUND

In new generations of implants wireless energy transmission is used for supply of energy in connection with implants. To optimise the transfer efficiency of the wireless energy it is important to locate the patient's wireless energy receiver, typically subcutaneously implanted, in order to be able to put an exterior energy transmission device close to the implanted energy receiving device.

### SUMMARY OF THE INVENTION

The object of the present invention is to provide an inexpensive apparatus for accurate detection of a wireless energy receiving device subcutaneously implanted in a patient, and further to provide an apparatus with parts to be implanted that are relatively small.

In accordance with a first aspect of the present invention, there is provided an apparatus for detecting a wireless energy receiving device subcutaneously implanted in a patient to enable accurate positioning of a wireless energy transmission device outside the patient's body for the transmission of wireless energy to the energy receiving device. The apparatus comprises a magnetic device designed to be subcutaneously implanted in the patient at the energy receiving device to emit a local magnetic field extending through the energy receiving device and a portion of the patient's skin adjacent the energy receiving device, and a magnetic detector movable externally along the patient's body and capable of detecting the local magnetic field emitted by the magnetic device when the magnetic detector is in a position in front of the implanted energy receiving device, whereby the energy transmission device can be located to efficiently transmit wireless energy to the implanted energy receiving device.

Alternatively, the apparatus comprises a magnetic detector designed to be subcutaneously implanted in the patient at the energy receiving device, an exterior magnetic device movable along the patient's skin and adapted to emit a local magnetic field through the patient's skin, the magnetic detector being capable of detecting the local magnetic field when the movable magnetic device is moved to a position in which it is in front of the implanted energy receiving device, whereby the energy transmission device can be located to efficiently transmit wireless energy to the implanted energy receiving device.

Thus, the present invention provides an easy way of detecting the position of a wireless energy receiving device subcutaneously implanted in a patient, which enables accurate positioning of a wireless energy transmission device outside the patient's body for efficient transmission of wireless energy to the implanted energy receiving device by using magnetism that is detected by a semiconductor circuit. The energy receiving device is connected (via, e.g., an electrical conduit) to an implant, for example a food intake restriction apparatus, implanted inside the human body.

In the alternative embodiment where a magnetic detector is to be implanted, a sender capable of sending information about the magnetic detector detecting the local magnetic field may be implanted in the patient.

Preferably, the magnetic detector includes a semiconductor circuit. According to a preferred embodiment of the invention, the semiconductor circuit of the magnetic detector comprises at least one Hall-element. By using one or more Hall-elements, a special type of semiconductor known in the art, it is easy to locate the central axis of the emitted magnetic field. The magnetic detector suitably comprises several Hall-elements which are grouped around a central point in a triangular or square configuration. One important feature is that the Hall-elements are able to detect even a weak magnetic field emitted from the magnetic device. The described detection technique is simple, inexpensive and very accurate, and could be used for several different implants in combination with wireless energy transmission.

The implanted energy receiving device may be of the type that transforms the received energy into electrical pulses. In this case, the apparatus may further comprise a sender to be implanted in the patient for sending feedback information on the number of electrical pulses that have been provided by the energy receiving device.

The magnetic device may be a solenoid or a permanent magnet, which is sending out a magnetic field. If the magnetic device is placed outside the body, the magnetic detector placed inside the body should preferably also be capable of sending information about the position of the magnetic device, directly or indirectly correlated to the intensity of magnetism to outside the body. Alternatively, the efficiency of energy transfer is another way of locating the implanted wireless energy receiving device. Thus, the energy transmission device may transmit energy in the form of energy pulses and the implanted energy receiving device may be capable of sending information about the amount of energy received, in the form of the number of energy pulses received. This information is sent to the outside of the patient's body and may be used to optimise the energy transfer. Thus, the wireless energy transmission device outside the patient's body may be moved accordingly, if the pulses are getting more frequent or more sparse, respectively. The information can be used to interpret if the energy transmission is becoming better or worse. The number of pulses can also be used as feedback information to not transmit excess amount of energy to the energy receiving device. Alternately, the information on the number of energy pulses received may also be used to increase or decrease the amount of transmitted energy. Hence, the energy transmission can be adjusted in both directions, i.e., to increase or decrease the voltage.

Conveniently, the location of the wireless energy receiving device, subcutaneously implanted in a patient, may be visualised on a screen, by sound or by diodes.

In accordance with a second aspect of the present invention, there is provided a method for detecting a wireless energy receiving device subcutaneously implanted in a patient, the method comprising the steps of: implanting a magnetic device subcutaneously in the patient at the energy receiving device so that the magnetic device emits a local magnetic field extending through the energy receiving device and the adjacent skin portion of the patient; and moving an exterior magnetic detector along the patient's skin to a position where the local magnetic field emitted by the implanted magnetic device is detected by the magnetic detector. Then, the energy transmission device can be put in the position where the local magnetic field has been detected to efficiently transmit wireless energy to the subcutaneously implanted energy receiving device.

As an alternative, the method may comprise the steps of: implanting a magnetic detector subcutaneously in the patient at the energy receiving device; moving an exterior magnetic device along the patient's skin while it emits a local magnetic field extending through the adjacent skin portion; using the implanted magnetic detector to detect the local magnetic field when the magnetic device is moved to a position in which the local magnetic field extends through the implanted magnetic detector and energy receiving device. This alternative method may further comprise implanting a sender and using the sender to send information to outside the patient's body confirming when the implanted magnetic detector detects the local magnetic field emitted by the exterior magnetic device.

In accordance with a third aspect of the invention, there is provided a surgical method for treating a patient having a disease, comprising the steps of: insufflating the patient's abdomen with gas; placing at least two laparoscopical trocars in the patient's body; implanting an operable implant designed for treating reflux disease, urinary incontinence, impotence, anal incontinence or obesity in the abdomen by using surgical instruments through the trocars; subcutaneously implanting a wireless energy receiving device for supplying energy for use in the operation of the implant and a magnetic device at the energy receiving device for emitting a local magnetic field through the energy receiving device and the adjacent skin portion of the patient; postoperatively moving an exterior magnetic detector along the patient's body to a position in which the local magnetic field emitted by the implanted magnetic device is detected by the magnetic detector; bringing a wireless energy transmission device to the position in which the local magnetic field is detected; and transmitting wireless energy from the wireless energy transmission device to the implanted wireless energy receiving device.

As an alternative, the surgical method may comprise subcutaneously implanting a magnetic detector at the energy receiving device and post-operatively moving an exterior magnetic device emitting a local magnetic field along the patient's body to a position in which the local magnetic field emitted by the exterior magnetic device is detected by the implanted magnetic detector.

The above described apparatuses and methods may also be designed for treating reflux disease, urine incontinence, anal incontinence, obesity and impotence.

### BRIEF DESCRIPTION OF THE DRAWINGS

FIG. 1 shows a connection diagram for a magnetic detector according to one aspect of the present invention.

FIG. 2 schematically illustrates in a diagram the position relative to the magnet as a function of the sensor (i.e., detector) output according to one aspect of the present invention.

FIG. 3 is a schematic view of an embodiment where a magnetic device is subcutaneously implanted in the patient, and a magnetic detector movable externally along the patient's body.

FIG. 4 is a schematic view of an embodiment where a magnetic detector is subcutaneously implanted in the patient and an exterior magnetic device is movable along the patient's skin.

FIG. 5 is a schematic view of a band with a cavity defining a restriction opening for use in accordance with one aspect the present invention, designed for treating reflux disease, urine incontinence, anal incontinence or obesity.

FIG. 6 illustrates an embodiment according to the present invention using several Hall-elements as the magnetic detecting device.

### DETAILED DESCRIPTION OF THE INVENTION

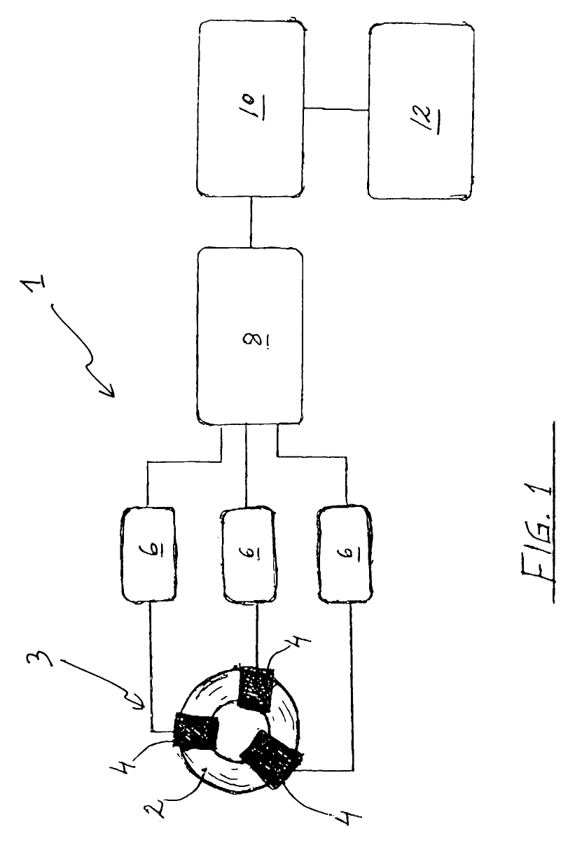

FIG. 1 shows a connection circuit 1 for a magnetic detector 3 according to the present invention. The magnetic device implanted inside a human body is a ring-magnet 2, which can be a solenoid or a permanent magnet. Located outside the body is magnetic detector 3, which is comprised of three linear magnetic field sensors 4 (such as Hall-elements or the like) grouped in a triangular configuration. Sensors 4 are connected to signal-conditioning amplifiers 6, which in turn, are connected to an A/D-converter 8. A microprocessor 10 is then connected to A/D-converter 8. To visually display the output signals of sensors 4, a display-device 12 is then connected to microprocessor 10.

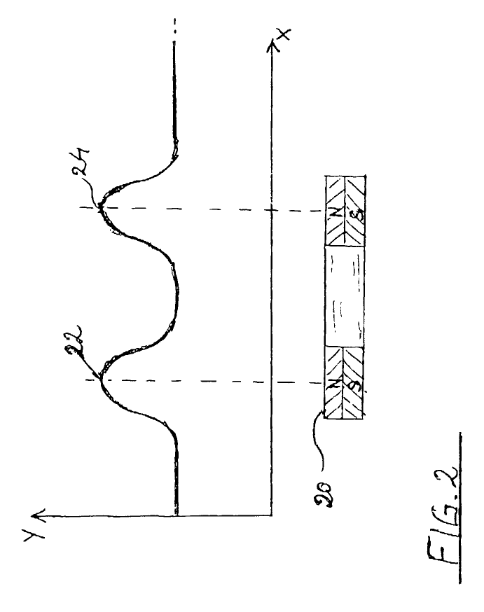

The graph shown in FIG. 2 illustrates, in principle, how the information obtained by detector 3 can be presented. On the X-axis in the graph is the position of detector 3 relative to magnet 2. On the Y-axis is the combined output of sensors 4. Thus, the graph of FIG. 2 shows the position “X” of detector 3 relative to magnet 2 as a function of detector 3's output “Y”. To illustrate this method of sensing, a ring-magnet 20 is shown relative to the graph of FIG. 2. Ring-magnet 20 is shown in cross-section to show the positions of its magnetic northpole N and southpole S, respectively. FIG. 2 depicts the case where magnetic detector 3 (not shown in FIG. 2) has been centered in front of ring-magnet 20 and where all of the sensors 4 produce a maximum output which is shown as peaks 22, 24 in the graph of FIG. 2. Sensors 4 are connected (e.g., by connection circuit 1 shown in FIG. 1) to display device 12, which may display the graph shown in FIG. 2, or alternatively, a numeral result from the measurements taken by sensors 4.

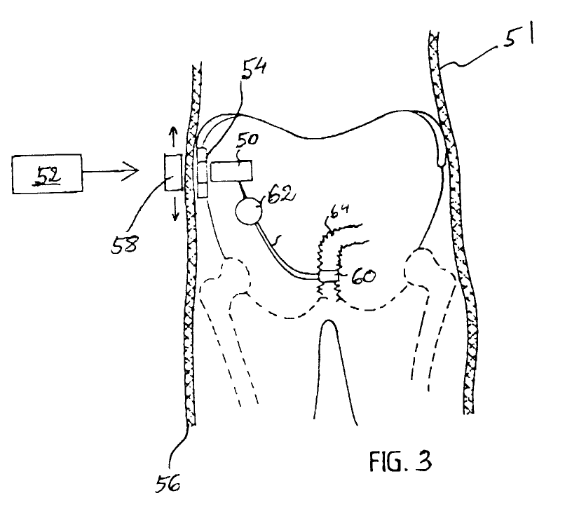

FIG. 3 shows an embodiment of the apparatus of the present invention for detecting a wireless energy receiving device 50 subcutaneously implanted in a patient 51 suffering from anal incontinence to enable accurate positioning of a separate wireless energy transmission device 52 outside patient 51's body for the transmission of wireless energy to energy receiving device 50. An operation device 62 is adapted to operate an implanted artificial sphincter 60 applied to the patient's rectum 64. Energy receiving device 50 powers operation device 62 with energy received from the energy transmission device 52. The apparatus of the present invention is also comprised of a magnetic device 54 subcutaneously implanted in patient 51 by the energy receiving device 50. Magnetic device 54 emits a local magnetic field extending through the energy receiving device 50 and a portion of patient 51's skin 56 adjacent to energy receiving device 50. The apparatus of the present invention is further comprised of an external, separate magnetic detector 58 that may be manually moved along the patient 51's body to detect the local magnetic field emitted by implanted magnetic device 54. Magnetic detector 58 detects the local magnetic field when it is positioned in front of implanted energy receiving device 50. When this position has been determined, energy transmission device 52 can be located in the same position to efficiently transmit wireless energy to implanted energy receiving device 50.

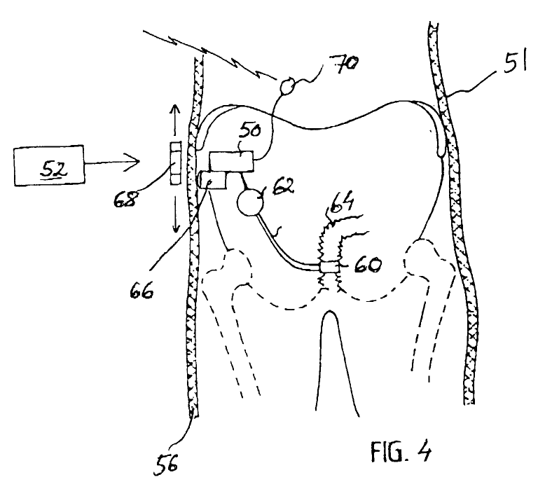

FIG. 4 shows a modification of the embodiment of FIG. 3, and is comprised of a magnetic detector 66 subcutaneously implanted in patient 51 at energy receiving device 50. An external separate magnetic device 68 may be manually moved along the patient 51's body while emitting a local magnetic field through the patient 51's skin 56. Magnetic detector 66 is capable of detecting the local magnetic field when movable magnetic device 68 is moved to a position in front of implanted energy receiving device 50. A sender 70 is implanted in patient 51 and sends information about the status of magnetic detector 66. Thus, when magnetic detector 66 detects the local magnetic field emitted by external magnetic device 68, sender 70 sends information confirming that magnetic device 68 is in a proper position for energy transmission. When this position has been determined, energy transmission device 52 can be placed in the same position to efficiently transmit wireless energy to the implanted energy receiving device 50.

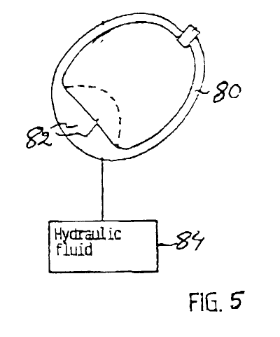

FIG. 5 shows an example of artificial sphincter 60, which is comprised of a band 80 formed into a loop around the patient's rectum (not shown in FIG. 5). Band 80 has a cavity 82 which can be inflated by supplying hydraulic fluid thereto, to close the rectum, and be deflated by withdrawing hydraulic fluid therefrom, to open the rectum. A hydraulic operation device 84 for operating band 80 is powered with energy from implanted energy receiving device 50. This type of band may also be used as an artificial sphincter for treating patient's suffering from heartburn and reflux disease or urinary incontinence, when combined with the apparatus of the present invention. Furthermore, band 80 may be used for forming an adjustable constricted stoma opening in the stomach or esophagus of an obese patient to treat obesity or for restricting the penile exit blood flow to treat an impotent patient, when combined with the apparatus of the invention.

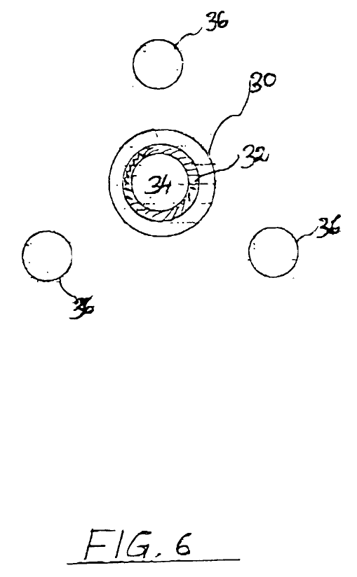

FIG. 6 shows an embodiment of the apparatus of the present invention where magnetic detector 58 suitably comprises several Hall-elements 36 which are grouped around a central point in a triangular or square configuration. In this embodiment, the magnetic device positioned by the implanted energy receiving device 30 is preferably a ring-shaped magnet 32 surrounding the centre 34 of energy receiving device 30. The magnetic detecting device 58 arranged outside patient 51's body comprises the three symmetrically arranged Hall-elements 36. When Hall-elements 36 are placed symmetrically above and around ring-shaped magnet 32, i.e., when ring-shaped magnet 32 is in the center 34 of energy receiving device 30, information is sent, directly or indirectly correlated to the intensity of the magnetism, about the location of the energy receiving device 30.

Although the present invention has been described in terms of a particular embodiment and process, it is not intended that the invention be limited to that embodiment. Modifications of the embodiment and process within the spirit of the invention will be apparent to those skilled in the art. The scope of the invention is defined by the claims that follow.

What is claimed is:

1. An apparatus for detecting a wireless energy receiving device adapted to be subcutaneously implanted in a patient to enable accurate positioning of a wireless energy transmission device outside the patient's body for transmission of wireless energy to the energy receiving device, comprising a magnetic device designed to be subcutaneously implanted in the patient at the energy receiving device for emitting a local magnetic field extending through the energy receiving device and a portion of the patient's skin adjacent to the energy receiving device, and a magnetic detector movable externally along the patient's body and capable of detecting said local magnetic field emitted by said magnetic device when said magnetic detector is in a position in front of the implanted energy receiving device, whereby the wireless energy transmission device can be put in said position to efficiently transmit wireless energy to the implanted energy receiving device.

2. An apparatus according to claim 1, wherein said magnetic device is a solenoid or a permanent magnet.

3. An apparatus according to claim 1, wherein said magnetic detector comprises a semiconductor circuit.

4. An apparatus according to claim 3, wherein said semiconductor circuit of said magnetic detector comprises at least one Hall-element.

5. An apparatus according to claim 4, wherein said magnetic detector comprises several Hall-elements grouped around a central point in a triangular or square-configuration.

6. An apparatus according to claim 1, wherein the energy receiving device transforms the received energy into electrical pulses, and further comprising a sender to be implanted in the patient for sending feedback information on the number of said electrical pulses to outside the patient's body.

7. An apparatus for detecting a wireless energy receiving device adapted to be subcutaneously implanted in a patient to enable accurate positioning of a wireless energy transmission device outside the patient's body for transmission of wireless energy to the energy receiving device, comprising a magnetic detector designed to be subcutaneously implanted in the patient at said energy receiving device, an exterior magnetic device movable along the patient's skin and adapted to emit a local magnetic field through the patient's skin, said magnetic detector being capable of detecting said local magnetic field when said movable magnetic device is moved to a position in which it is in front of said implanted energy receiving device, whereby the energy transmission device can be put in said position to efficiently transmit wireless energy to the implanted energy receiving device.

8. An apparatus according to claim 7, further comprising a sender adapted to be implanted in the patient and capable of sending information about said magnetic detector detecting said local magnetic field.

9. An apparatus according to claim 7 wherein said magnetic device is a solenoid or a permanent magnet.

10. An apparatus according to claim 7, wherein said magnetic detector comprises a semiconductor circuit.

11. An apparatus according to claim 10, wherein said semiconductor circuit of said magnetic detector comprises at least one Hall-element.

12. An apparatus according to claim 7, wherein the energy receiving device transforms the received energy into electrical pulses, and further comprising a sender adapted to be implanted in the patient for sending feedback information on the number of said electrical pulses to outside the patient's body.

13. A method for detecting a wireless energy receiving device adapted to be subcutaneously implanted in a patient, the method comprising the steps of:
implanting a magnetic device subcutaneously in the patient at the energy receiving device so that the magnetic device emits a local magnetic field extending through the energy receiving device and the adjacent skin portion of the patient; and
moving an exterior magnetic detector along the patient's skin to a position where the local magnetic field emitted by the implanted magnetic device is detected by the magnetic detector.

14. A method for detecting a wireless energy receiving device adapted to be subcutaneously implanted in a patient, the method comprising the steps of:
implanting a magnetic detector subcutaneously in the patient at the energy receiving device;
moving an exterior magnetic device along the patient's skin while it emits a local magnetic field extending through the adjacent skin portion; and
using the implanted magnetic detector to detect the local magnetic field when the magnetic device is moved to a position in which the local magnetic field extends through the implanted magnetic detector and energy receiving device.

15. A method according to claim 14, further comprising implanting a sender and using the sender to send information to outside the patient's body confirming when the implanted magnetic detector detects the local magnetic field emitted by the exterior magnetic device.

16. A surgical method for treating a patient having a disease, comprising the steps of:
insufflating the patient's abdomen with gas;
placing at least two laparoscopical trocars in the patient's body;
implanting an operable implant designed for treating reflux disease, urine incontinence, impotence, anal incontinence or obesity in the abdomen by using surgical instruments through the trocars;
subcutaneously implanting a wireless energy receiving device for supplying energy for use in the operation of the implant and a magnetic device at the energy receiving device for emitting a local magnetic field through the energy receiving device and the adjacent skin portion of the patient;
post-operatively moving an exterior magnetic detector along the patient's body to a position in which the local magnetic field emitted by the implanted magnetic device is detected by the magnetic detector;
bringing a wireless energy transmission device to the position in which the local magnetic field is detected; and
transmitting wireless energy from the wireless energy transmission device to the implanted wireless energy receiving device.

17. A surgical method for treating a patient having a disease, comprising the steps of:
insufflating the patient's abdomen with gas;
placing at least two laparoscopical trocars in the patient's body;
implanting an operable implant designed for treating reflux disease, urine incontinence, impotence, anal incontinence or obesity in the abdomen by using surgical instruments through the trocars;
subcutaneously implanting a wireless energy receiving device for supplying energy for use in the operation of the implant and a magnetic detector at the energy receiving device;
post-operatively moving an exterior magnetic device emitting a local magnetic field along the patient's body to a position in which the local magnetic field emitted by the exterior magnetic device is detected by the implanted magnetic detector;
bringing a wireless energy transmission device to the position in which the local magnetic field is detected; and
transmitting wireless energy from the wireless energy transmission device to the implanted wireless energy receiving device.
